(19) United States
(12) Plant Patent Application Publication
(10) Pub. No.: US 2006/0070156 P1
(43) Pub. Date: Mar. 30, 2006

(54) Geranium plant named 'Lavender Dot'

(76) Inventor: Laura Bartlett McCarthy, Sudbury, MA (US)

Correspondence Address: J.P. Bartlett Co Inc, 578 Boston Post Rd, Sudbury, MA, 01776, US

(21) Appl. No.: 10/950,297

(22) Filed: Sep. 27, 2004

(51) Int. Cl. A01H 5/00

(52) U.S. Cl. PLT/329

(57) ABSTRACT

This invention is a new and distinct cultivar of a zonal geranium (Pelagronium×hortorum) named ‘Lavender Dot’ the cultivar has a brilliant array of colors within the plant's light lavender pink blossom and dark green foliage on a stable, full, well branched plant.

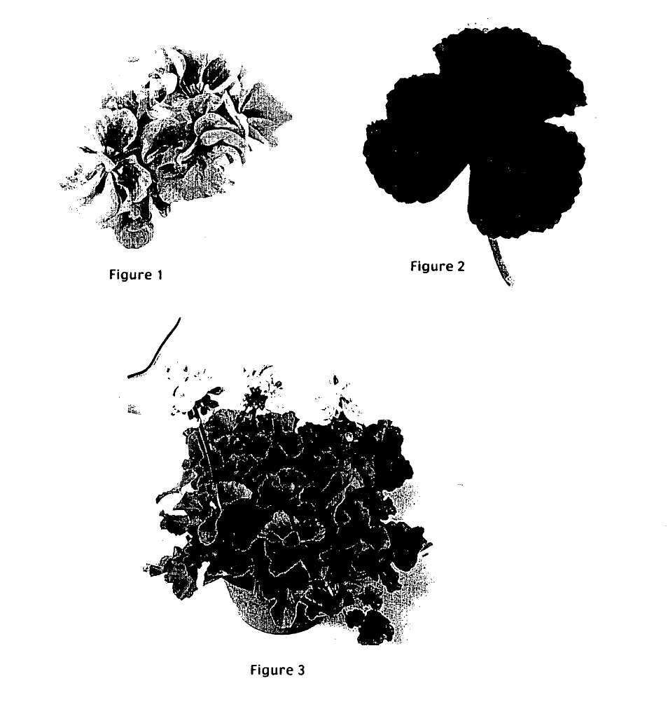

### BOTANICAL CLASSIFICATION/CULTIVAR DESIGNATION

Pelargonium.times.hortorum cultivar ‘Lavender Dot’

### BACKGROUND OF THE INVENTION

The cultivar was developed from a controlled breeding program conducted at the J.P. Bartlett Company. The company used a non-patented cultivar designated #Puritan 2 as the male parent and a non-patented cultivar designated Quaker as the female parent in a cultivated area to create ‘Lavender Dot’. ‘Lavender Dot's’ plant habit is symmetrical with excellent basal as well as a lateral branching habit. Asexual propagation of the new cultivar by cuttings at the location previously stated has shown that the unique features of this new geranium are stable and that the plant reproduces true to type in successive generations of asexual propagation.

### SUMMARY OF THE INVENTION

The plants growth habit is compact to medium in vigor. It is a full plant that performs well in both a greenhouse and in a natural environment such as a garden. It will grow more compact under drier conditions but has the ability to be a larger and more vigorous plant with sufficient water and partial shade. The blossoms are tolerant to rain, not turning brown and rotting after a day or so of straight rain. Lavender Dot buds early and is consistent with a large and longer lasting blossom than many other cultivars known to me.

On average, the plant itself can get to over 309 millimeters tall, not including the stem or flower. The free branching nature of Lavender Dot allows the plants girth to grow and flourish. The width of a single plant is measured to be 495 millimeters and growing. The striking two tone blossom color and dark green leaf color separates itself from any other known to me. The semi-double blossom is a light lavener pink with a darker pink to each petal. ‘Lavender Dot's’ stem is a distinguished light green, while the leaf produces a unique variation of colors all in its own. ‘Lavender Dot's’ standout performance is regarded highly due to it's excellent growth habit, size, blossom and leaf. The new cultivar of the present invention is being marketed under the BARTLETT'S trademark.

### BRIEF DESCRIPTION OF THE PHOTOGRAPH

Accompanying this application is a photographed picture of the plant and its' aspects.

1. Figure one shows the large, semi-double blossom and the purple/violet color described in the summary.

2. Figure two enables us to see the medium green leaf with the medium green zone.

3. Figure three shows the overall plant. This is a single plant from a cutting in a four-inch pot. The height, width, and growth habit are clearly seen here.

### DETAILED BOTANICAL DESCRIPTION

The following observations and measurements describe asexual reproductions of ‘Lavender Dot’ grown in greenhouses located in Sudbury, Mass., USA. The plant history was taken on 3 month old plants grown in 7.5″ pots, blossomed under natural light in a greenhouse and color readings were taken in the greenhouse in Sudbury, Mass. under ambient light. Color references are primarily to the Pantone Color Selector Chart 1000/coated 1995. The color values were determined on Oct. 22, 2003 under natural light conditions of 200 foot-candles.
Classification:
   Botanical.—Pelargonium×hortorum Bailey, cv. ‘Lavender Dot’.
Inflorescence:
A. Umbel:
   Average diameter.—Approximately 9 to 11.25 cm.
   Average depth.—Approximately 4.9 to 6.7 cm.
   Peduncle length.—Approximately 14.5 to 16.3 cm.
   Pedicel length.—Approximately 2.7 to 3.1 cm.
   Number of umbels per plant.—When grown in a 7.5″ pot at 8 weeks after the sticking of a rooted cutting, there commonly are approximately 7 to 9 umbels per plant.
   Number of florets/umbel.—When grown in a 7.5″ pot at 8 weeks, approximately 21 to 23 florets per umbel commonly are formed.
B. Corolla:
   Buds.—Elliptical to round in configuration, initially light green and changing to light pink at the time of opening, and approximately 11 to 14 mm. in length and approximately 5 to 7 mm. in width.
   Pedicels.—Commonly approximately 4.5 to 6 cm in length and curved which commonly causes the buds to point substantially downward.
   Average diameter.—Approximately 4 to 6 cm.
   Form.—Commonly semi-double with a t least two petaloids.
   Petals.—Spatulate shaped, satiny and smooth. Commonly approximately 9 to 11 petals are present per floret.
   Number of petaloids.—Commonly forms approximately 2 to 5 petaloids per floret.
   Color.—General tonality from a distance of three meters: light pink with dark pink center.
   Color of upper petals.—Pantone 1895C.
   Markings of upper petals.—Dark pink spot at base of petal Pantone 1915C.
C. Reproductive organs:
   Androecium.—5 fertile anthers, with whitish filaments, orange pollen the lower side of the mature (open) anther is dark brown.
   Gynoecium.—5 lobed pale pink stigma, Pantone 182C, greenish style; one pistil per flower.
   Fertility/Seed set.—Commonly does not produce fruits in the absence of mechanical fertilization.
   Spring flowering response period.—Approximately 5 to 8 weeks from rooted cuttings under standard greenhouse conditions.
   Outdoor flower production.—Freely flowering under outdoor growing conditions with substantially continuous blooming.
   Durability.—Ships well.
   Pest/Disease resistance/susceptibility.—No susceptibility to pests or diseases noted to date.
Plant:
A. Foliage: Medium green with slight zonation. The leaves are pubescent on the upper surface.
   Length.—3.5 to 4.2 cm.
   Width.—5.2 to 6.1 cm.
   Form.—Reniform, with a cordate base.
   Margin.—Irregularly crenate.
   Ribs and veins.—Palmate 5.1 to 5.9 cm. long.
   Color.—Upper side is Pantone 357C. Under side is Pantone 349C.
   Petioles.—Commonly approximately 2.5 to 5 cm.
A. General appearance and form:
   Internode length.—Commonly varies from approximately 0.6 to 0.9 cm.
   Branching pattern.—Freely basal branching as well as lateral branching. No pinching is required to obtain self-branching. A self-branching growth habit is observed in the absence of a growth regulator.
   Height.—Approximately 19 to 24 cm. above a 7.5″ pot at 8 weeks under standard greenhouse conditions.

1. J.P. Bartlett Co., Inc has bred a new and distinct geranium plant as shown and described
